(19) United States
(12) Patent Application Publication
(10) Pub. No.: US 2025/0205458 A1
(43) Pub. Date: Jun. 26, 2025

(54) METHODS FOR DETERMINING A POSITION OF A FIRST MEDICAL DEVICE WITH RESPECT TO A SECOND MEDICAL DEVICE, AND RELATED SYSTEMS AND MEDICAL DEVICES

(71) Applicant: Boston Scientific Medical Device Limited, Galway (IE)

(72) Inventors: Steven Kinio, Pickering (CA); Gareth Davies, Toronto (CA); Laurentiu Murtescu, Vaughn (CA)

(21) Appl. No.: 19/075,089

(22) Filed: Mar. 10, 2025

Related U.S. Application Data
(63) Continuation of application No. 18/342,353, filed on Jun. 27, 2023, now Pat. No. 12,246,148.
Application No. 18/342,353 is a division of application No. 16/720,212, filed on Dec. 19, 2019, now Pat. No. 11,724,070.

(51) Int. Cl. A61M 25/06; A61B 17/32; A61B 17/34; A61B 90/00

(52) U.S. Cl. CPC A61M 25/0606; A61B 17/32; A61B 17/34; A61B 17/3403; A61B 2090/0811; A61M 2205/0238; A61M 2205/0288; A61M 2205/3306; A61M 2205/3317; A61M 2205/50; A61M 2205/502; A61M 2205/587

(57) ABSTRACT

A system of medical devices includes a first medical device, a second medical device, an RLC circuit, a processor, and an output device. The first medical device includes an elongate member having a lumen. The second medical device includes a needle advanceable through the lumen. The RLC circuit includes an excitation voltage source, a resistor, a capacitor, an inductor, and an output voltage sensor. The inductor includes a coil that is supported by the first medical device and wound around the lumen. As the needle is advanced through the lumen, an output voltage of the RLC circuit is an indicator of a longitudinal position of the needle with respect to the elongate member. The voltage sensor is configured to sense the output voltage, and the processor and output device are configured to generate an output that is an indicator of the longitudinal position of the needle.

### CROSS REFERENCE TO RELATED APPLICATIONS

The present application is a continuation of U.S. patent application Ser. No. 18/342,353, filed Jun. 27, 2023, which is a divisional of U.S. patent application Ser. No. 16/720,212, filed Dec. 19, 2019, now issued as U.S. Pat. No. 11,724,070, which are incorporated herein in their entirety.

### FIELD

This document relates to medical devices. More specifically, this document relates to methods for determining a position of a first medical device with respect to a second medical device, and related systems and medical devices.

### SUMMARY

The following summary is intended to introduce the reader to various aspects of the detailed description, but not to define or delimit any invention.

Systems of medical devices are disclosed. According to some aspects, a system of medical devices includes a first medical device including an elongate member. The elongate member has a proximal portion defining a proximal end, a distal portion defining a distal end, and a lumen extending longitudinally therethrough from the proximal end to the distal end. The system further includes a second medical device including a needle advanceable through the lumen from the proximal end towards the distal end. The system further includes at least a first RLC circuit including an excitation voltage source, a resistor, a capacitor, an inductor, and an output voltage sensor. The inductor includes a coil that is supported by the first medical device and wound around the lumen. As the needle is advanced through the lumen, an output voltage of the RLC circuit is an indicator of a longitudinal position of the needle with respect to the elongate member. The voltage sensor is configured to sense the output voltage of the RLC circuit and generate a sensor signal based on the output voltage. A processor is in communication with the voltage sensor and is configured to receive and process the sensor signal from the voltage sensor, and generate a processor signal based on the sensor signal. An output device is in communication with the processor and is configured to receive the processor signal and generate an output based on the processor signal. The output is an indicator of the longitudinal position of the needle with respect to the elongate member.

In some examples, the elongate member includes a sidewall extending longitudinally between the proximal end and the distal end, and radially between an inner surface and an outer surface, and the coil is embedded in the sidewall.

In some examples, the inductor further includes a magnetic core positioned radially inwardly of the coil and supported by the first medical device. The magnetic core can be embedded in the sidewall.

In some examples, the inductor further includes a magnetic core, and the magnetic core is provided by a magnetic coating on the needle. In some examples, the needle is fabricated from a magnetic material to provide a magnetic core of the inductor.

In some examples, the coil extends continuously from the proximal portion to the distal portion. In some examples, the coil is positioned within the distal portion, proximate the distal end.

In some examples, the system further includes a second RLC circuit including a second excitation voltage source, a second resistor, a second capacitor, a second inductor comprising a second coil, and a second output voltage sensor. The second coil can be supported by the first medical device, wound around the lumen, and spaced proximally from the coil of the first RLC circuit. As the needle is advanced through the lumen, an output voltage of the second RLC circuit can be an additional indicator of the longitudinal position of the needle with respect to the elongate member.

In some examples, the output device includes at least one of a light and a screen. The output can include illumination of the light when the needle is at a predetermined longitudinal position with respect to the elongate member. The output can include a GUI showing an image of the longitudinal position of the needle with respect to the elongate member.

Methods for determining a position of a first medical device with respect to a second medical device are also disclosed. According to some aspects, a method for determining a position of a first medical device with respect to a second medical device includes: a. advancing the second medical device into a lumen of the first medical device, from a proximal end of the first medical device towards a distal end of the first medical device; b. during step a., applying an excitation voltage to an RLC circuit associated with the first medical device, and sensing an output voltage of the RLC circuit, whereby the output voltage is an indicator of a longitudinal position of the second medical device with respect to the first medical device; and c. generating an output based on the output voltage, wherein the output is an indicator of the longitudinal position of the second medical device with respect to the first medical device.

In some examples, step a. includes passing the second medical device through a coil of an inductor of the RLC circuit.

In some examples, step c. includes generating an image of the longitudinal position of the second medical device with respect to the first medical device, and updating the image as the output voltage changes.

In some examples, the method further includes adjusting a position of the second medical device with respect to the first medical device based on the output.

Medical devices are also disclosed. According to some aspects, a medical device includes an elongate member having a proximal portion defining a proximal end, a distal portion defining a distal end, a sidewall extending longitudinally between the proximal end and the distal end and radially between an inner surface that and an outer surface, and a lumen defined by the inner surface and extending longitudinally through the elongate member from the proximal end to the distal end. The medical device further includes an inductor including a coil embedded in the sidewall and wound around the lumen. The coil is electrically connectable to an excitation voltage source, a resistor, a capacitor, and an output voltage sensor to form an RLC circuit.

In some examples, the inductor further includes a magnetic core positioned radially inwardly of the coil and supported by the elongate member. The magnetic core can be embedded in the sidewall.

In some examples, the coil extends continuously from the proximal portion to the distal portion. In some examples, the coil is positioned within the distal portion, proximate the distal end.

In some examples, the device includes a second inductor. The second inductor can include a second coil embedded in the sidewall and wound around the lumen. The second coil can be spaced proximally from the coil of the first RLC circuit. The second coil can be electrically connectable to a second excitation voltage source, a second resistor, and a second output voltage sensor to form a second RLC circuit.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of a first example system of medical devices, showing a first medical device, second medical device, and control unit, spaced apart and disconnected from each other;

FIG. 2 is a perspective view of the system of FIG. 1, showing the second medical device inserted into the first medical device, and the first medical device connected to the control unit;

FIG. 3 is a longitudinal cross section taken along line 3-3 in FIG. 2, schematically showing the control unit and related parts;

FIG. 4 is a simplified graph showing the change in output voltage as the second medical device of FIGS. 1 to 3 is advanced through the first medical device of FIGS. 1 to 3;

FIG. 5 is a cross-section similar to that of FIG. 3, showing a first medical device, second medical device, and control unit of a second example system;

FIG. 6 is a simplified graph showing the change in output voltage as the second medical device of FIG. 5 is advanced through the first medical device of FIG. 5;

FIG. 7 is a cross-section similar to that of FIG. 3, showing a first medical device, second medical device, and control unit of a third example system;

FIG. 8 is a cross-section similar to that of FIG. 3, showing a first medical device, second medical device, and control unit of a fourth example system;

FIG. 9 is a simplified graph showing the change in output voltage as the second medical device of FIG. 8 is advanced through the first medical device of FIG. 8;

FIG. 10 is a cross-section similar to that of FIG. 3, showing a first medical device, second medical device, and control unit of a fifth example system;

FIG. 11 is a simplified graph showing the change in output voltage as the second medical device of FIG. 10 is advanced through the first medical device of FIG. 10; and

FIG. 12 is a cross-section similar to that of FIG. 3, showing a first medical device, second medical device, and control unit of a sixth example system.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are systems of medical devices, where the systems include an RLC circuit (i.e. at least one RLC circuit). The RLC circuit can be used to determine a position of a first medical device of the system with respect to a second medical device of the system. For example, a system of medical devices can include a first medical device (e.g. a catheter in the form of a sheath or dilator) that in use is advanced into a patient's body to a target location (e.g. advanced via a femoral vein to a patient's heart) and a second medical device (e.g. a perforation device including a needle) that in use is passed through the first medical device towards the target location. The inductor of the RLC circuit can include a coil that that is wound so that as the second medical device is passed through the first medical device, it passes through the coil. In use, an excitation voltage can be applied to the RLC circuit, and an output voltage of the RLC circuit can be sensed and monitored. Passage of the second medical device through the coil can change the inductance of the inductor and thus change the output voltage of the RLC circuit (e.g. the output voltage can become non-resonant). The output voltage can thus be used as an indicator of a position of the second medical device with respect to the first medical device. Accordingly, by sensing the output voltage, it can be determined, for example, whether a perforating tip of a needle is approaching a distal end of the catheter, or has passed beyond a distal end of the catheter. This can facilitate ease of use of the medical devices, and enhance patient safety.

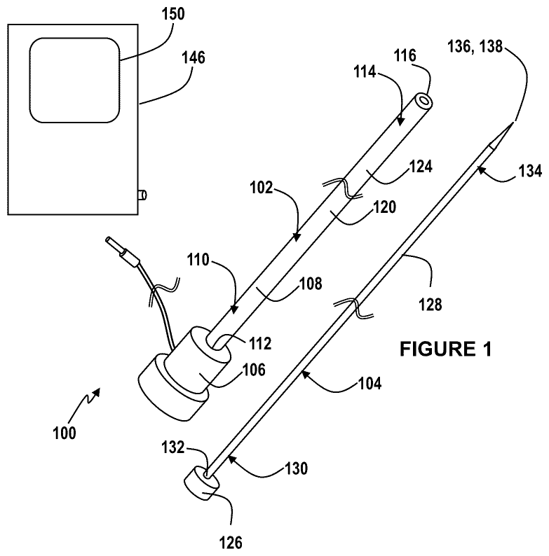

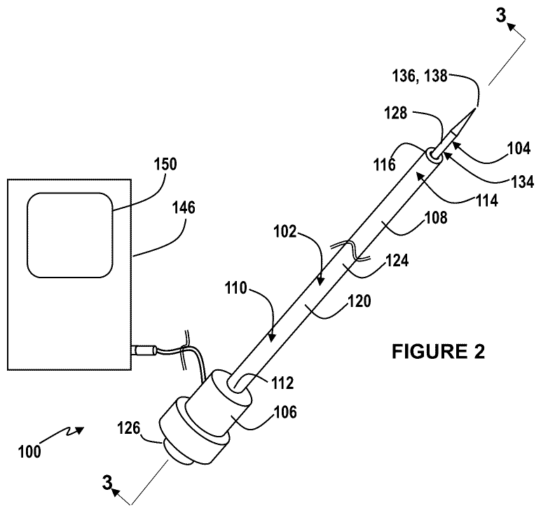

Referring now to FIGS. 1 and 2, a first example system 100 of medical devices is shown. In the example shown, the system 100 includes a first medical device 102 in the form of a catheter, and a second medical device 104 in the form of a perforation device. The catheter can be, for example, a sheath, a dilator, or an alternative device that is intended for use by passing another medical device therethrough (e.g. coaxially therethrough). The perforation device can be, for example, a mechanical perforation device, or a radiofrequency (RF) perforation device. In alternative examples, the second medical device can be alternative type of medical device that is intended for use by being passed through another medical device.

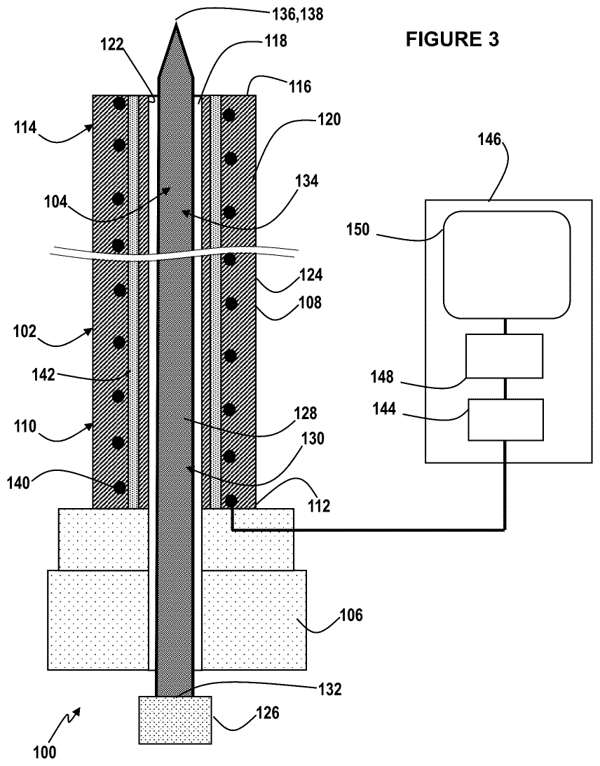

Referring also to FIG. 3, in the example shown, the first medical device 102 includes a hub 106 and an elongate member 108 extending from the hub 106. The elongate member 108 has a proximal portion 110 defining a proximal end 112 of the elongate member 108, a distal portion 114 defining a distal end 116 of the elongate member 108, and a lumen 118 (shown in FIG. 3) extending longitudinally through the elongate member 108 from the proximal end 112 to the distal end 116. The elongate member 108 includes a sidewall 120, which extends longitudinally between the proximal end 112 and the distal end 116, and radially between an inner surface 122 (shown in FIG. 3) that defines the lumen 118, and an outer surface 124. The sidewall 120 can be made from an electrically insulating material, such as an electrically insulating polymer (e.g. polyurethane).

Referring still to FIGS. 1 to 3, in the example shown, the second medical device 104 includes a hub 126 and a needle 128 extending from the hub 126. The needle 128 is electrically conductive (e.g. is metallic). The needle 128 has a proximal portion 130 defining a proximal end 132 of the needle 128, and a distal portion 134 defining a distal end 136 of the needle 128. The distal end 136 of the needle 128 includes a perforating tip 138. As shown in FIG. 2, the needle 128 is advanceable through the hub 106 and through the lumen 118, from the proximal end 112 of the elongate member 108 towards the distal portion 114 of the elongate member 108, to position the perforating tip 138 of the needle 128 proud of the distal end 116 of the elongate member 108.

Referring to FIG. 3, the system 100 further includes an inductor, which in the example shown includes a coil 140, and a magnetic core 142. The inductor is part of an RLC circuit. RLC circuits in general are known in the art, and are not described in detail herein. Briefly, in addition to the inductor, the RLC circuit further includes a resistor and a capacitor, as well as an excitation voltage source and an output voltage sensor. The parts of the RLC circuit, other than the inductor, are shown collectively at 144 in FIG. 3.

Referring still to FIG. 3, in the example shown, the coil 140 is supported by the first medical device 102, and is wound around the lumen 118. As used herein, the term “supported by” indicates that the coil is integral with, embedded in, connected to, mounted to, adhered to, affixed to, or otherwise secured to the first medical device 102, so that the coil moves with the first medical device 102. In the example shown, the coil is embedded in the sidewall 120. The coil 140 can be, for example, a wire such as copper wire.

Referring still to FIG. 3, in the example shown, the magnetic core 142 is positioned within the coil 140 and is also supported by the first medical device 102. In the example shown, the magnetic core 142 is embedded in the sidewall 120, radially inwardly of the coil 140. The magnetic core 142 can be, for example, a magnetic tape that is wound around the lumen 118 and embedded in the sidewall 120.

Referring still to FIG. 3, in the example shown, the coil 140 and magnetic core 142 extend continuously from the proximal portion 110 to the distal portion 114 of the elongate member 108. In alternative examples, a coil and/or magnetic core can extend along less than the entire length of the elongate member, such as along a majority of the length of the elongate member, or along only a small section of the elongate member. For example, a coil and magnetic core can be relatively short in length, and can be positioned within the distal portion of the elongate member, proximate the distal end (e.g. right at the distal end, or slightly proximal of the distal end).

Referring still to FIG. 3, the system further includes a control unit 146. The control unit 146 houses the components of the RLC circuit other than the inductor (i.e. the excitation voltage source, a resistor, a capacitor, and an output voltage sensor, shown at 144), which are electrically connected to the coil 140 to complete the RLC circuit. The coil 140 can optionally be removably electrically connected to the control unit 146, for example with a male connector (shown in FIG. 1) that can connect to a female connector of the control unit 146.

The control unit further 146 houses a processor 148, and an output device 150, described in further detail below

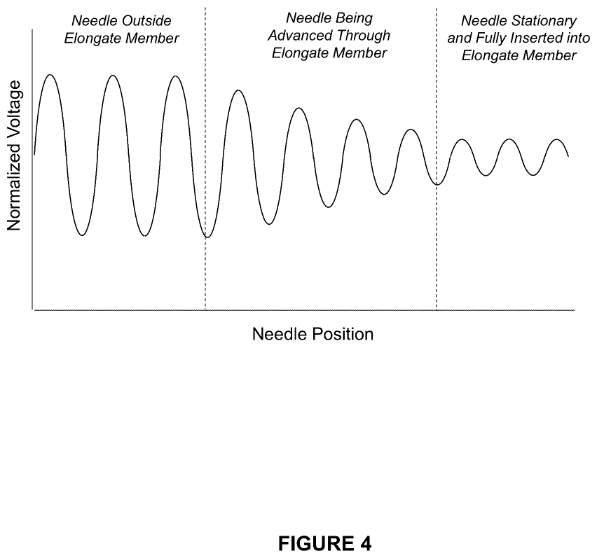

In use, an excitation voltage can be applied to the RLC circuit from the excitation voltage source as the needle 128 approaches and is advanced through the lumen 118. The excitation voltage can be tuned so that, in the absence of the needle 128 in the lumen 118, the circuit is resonant. The output voltage of the RLC circuit can be sensed as the needle 128 approaches and is advanced through the lumen 118. As the needle 128 enters and is advanced in the lumen 118, the inductance of the RLC circuit will change due to the presence of the needle 128. As shown in FIG. 4, the change in inductance will cause the output voltage of the RLC circuit to change—i.e. it can become non-resonant. The output voltage can thus be used as an indicator of the longitudinal position of the needle 128 with respect to the elongate member 108. For example, the output voltage can be an indicator of when the perforating tip 138 is well shrouded within the elongate member 108 (and thus when a patient's anatomy is protected from being perforated by the needle 128), or when the perforating tip 138 is at the distal end 116 of the elongate member 108 (i.e. when the needle 128 is ‘primed’ for use), or when the perforating tip 138 has passed beyond the distal end 116 of the elongate member 108 and is exposed (and thus when a patient's anatomy is not protected from being perforated by the needle 128).

Referring back to FIG. 3, the voltage sensor of the RLC circuit is in communication with the processor 148, and the processor 148 is in communication with the output device 150. The output voltage sensor senses the output voltage of the RLC circuit, and can generate a signal (referred to herein as a “sensor signal”) based on the sensed output voltage. The processor 148 can include various components (e.g. an analog to digital converter, isolation circuitry, and a microcontroller), and is configured to receive and process the signal from the output voltage sensor, and to generate a signal (referred to herein as a “processor signal”) based on the sensor signal. The output device 150 can receive the processor signal, and can generate an output based on the processor signal.

In some examples, the output device 150 can include a light, and the processor signal can cause illumination of the light, or a change in color of the light. For example, when the sensor signal indicates that the output voltage has reached a predetermined value that corresponds to the perforating tip 138 being at a predetermined longitudinal position with respect to the elongate member 108 (e.g. the perforating tip being at the distal end 116 of the elongate member 108), the processor 148 can signal the output device 150 to change the color of the light from green to red.

In some examples, the output device 150 can include a screen that shows a graphical user interface (GUI). The processor signal can cause the output device 150 to generate an image showing the longitudinal position of the needle 128 within the elongate member 108. For example, as the needle 128 is advanced through lumen 118 and the output voltage changes, the image can change based on the output voltage.

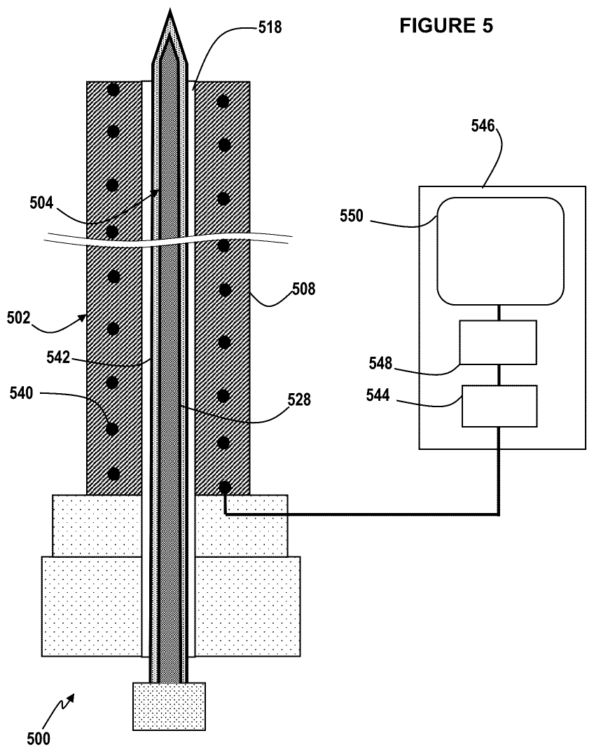

Referring now to FIG. 5, an alternative example system is shown. In the example of FIG. 5, features that are like those of FIGS. 1 to 3 will be referred to with like reference numerals, incremented by 400.

The system 500 of FIG. 5 is similar to the system 100 of FIGS. 1 to 3, and includes a first medical device 502 in the form of a catheter having an elongate member 508 with a lumen 518, a second medical device 504 including a needle 528 that is advanceable through the lumen 518, and a control unit 546, which houses a processor 548 and an output device 550. The system 500 further includes an inductor, which is part of an RLC circuit. The RLC circuit further includes an excitation voltage source, a resistor, a capacitor, and an output voltage sensor, which are shown collectively at 544 in FIG. 5. In the example of FIG. 5, the inductor includes a coil 540, and a magnetic core 542; however the magnetic core 542 of the inductor is provided by the needle 528, which includes a coating of a magnetic material (e.g. a coating of epoxy containing suspended ferrites or magnetic tape).

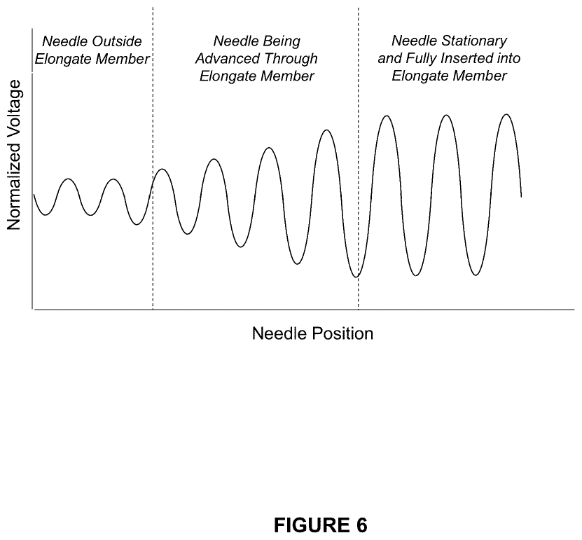

In use, an excitation voltage can be applied to the RLC circuit from the excitation voltage source as the needle 528 approaches and is advanced through the lumen 518. The excitation voltage can be tuned so that, when the needle 528 is fully inserted into the elongate member 508, the circuit is resonant. The output voltage of the RLC circuit can be sensed as the needle 528 approaches and is advanced through the lumen 518. As the needle 528 enters and is advanced in the lumen 518, the inductance of the RLC circuit will change due to the presence of the needle 528. As shown in FIG. 6, the change in inductance will cause the output voltage of the RLC circuit to change—i.e. it will become resonant. Similarly to the example of FIGS. 1 to 3, the output voltage can thus be used as an indicator of the longitudinal position of the needle 528 with respect to the elongate member 508.

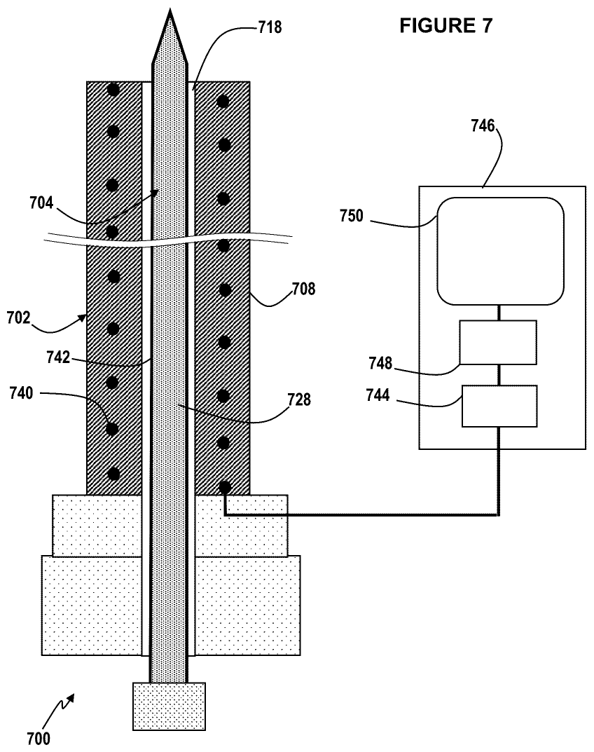

Referring now to FIG. 7, another alternative example system is shown. In the example of FIG. 7, features that are like those of FIGS. 1 to 3 will be referred to with like reference numerals, incremented by 600.

The system 700 of FIG. 7 is similar to the system 500 of FIG. 5, and includes a first medical device 702 in the form of a catheter having an elongate member 708 with lumen 718, a second medical device 704 including a needle 728 that is advanceable through the lumen 718, and a control unit 746, which houses a processor 748 and an output device 750. The system 700 further includes an inductor, which is part of an RLC circuit. The RLC circuit further includes an excitation voltage source, a resistor, a capacitor, and an output voltage sensor, which are shown collectively at 744 in FIG. 7. Similarly to the example of FIG. 5, the inductor includes a coil 740, and a magnetic core 742 that is provided by the needle 728; however, in the example of FIG. 7, the needle 728 is fabricated from a magnetic material, such as silicon steel, manganese-zinc ferrite, iron, etc., so that the needle 728 as a whole is magnetic.

The system of FIG. 7 can be operated similarly to the system of FIG. 5—i.e. the excitation voltage can be tuned so that, when the needle 728 is fully inserted into the elongate member 708, the circuit is resonant. The output voltage can thus be used as an indicator of the longitudinal position of the needle 728 with respect to the elongate member 708.

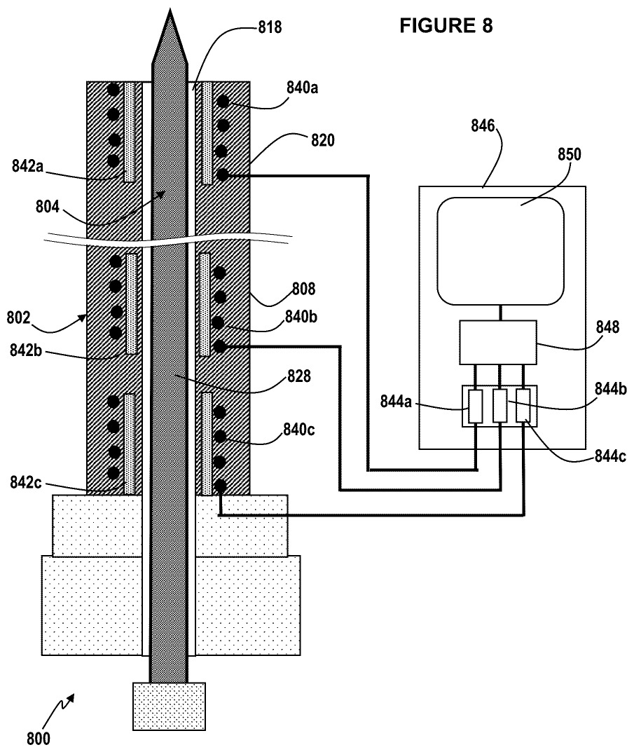

Referring now to FIG. 8, another alternative example system is shown. In the example of FIG. 8, features that are like those of FIGS. 1 to 3 will be referred to with like reference numerals, incremented by 700.

The system 800 of FIG. 8 is similar to the system 100 of FIGS. 1 to 3, and includes a first medical device 802 in the form of a catheter having a lumen 818, a second medical device 804 including a needle 828 that is advanceable through the lumen 818, and a control unit 846, which houses a processor 848 and an output device 850.

The system 800 of FIG. 8 includes a plurality of RLC circuits (three of which are shown). Each RLC circuit includes an inductor that includes a coil and a magnetic core (i.e. the first RLC circuit includes a first coil 840a and a first magnetic core 842a; the second RLC circuit includes a second coil 840b and a second magnetic core 842b; and the third RLC circuit includes a third coil 840c and a third magnetic core 842c). Each coil 840a, 840b, 840c is embedded in the sidewall 820, and each magnetic core 842a, 842b, 842c is embedded in the sidewall 820 radially inwardly of the coils 840a, 840b, 840c, respectively. The inductors are longitudinally spaced apart so that the second inductor is spaced proximally from the first inductor, and the third inductor is spaced proximally from the second inductor. The first RLC circuit further includes an excitation voltage source, a resistor, a capacitor, and an output voltage sensor, which are shown collectively at 844a in FIG. 8; the second RLC circuit further includes an excitation voltage source, a resistor, a capacitor, and an output voltage sensor, which are shown collectively at 844b in FIG. 8; and the third RLC circuit further includes an excitation voltage source, a resistor, a capacitor, and an output voltage sensor, which are shown collectively at 844c in FIG. 8.

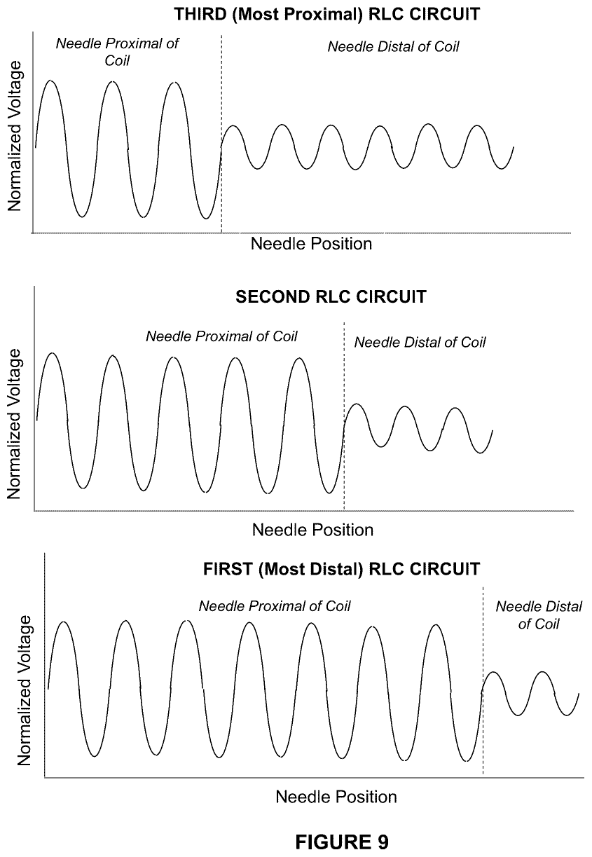

In use, an excitation voltage can be applied to each RLC circuit from the respective excitation voltage source as the needle 828 approaches and is advanced through the lumen 818. In use, each RLC circuit can be tuned so that a drop in signal amplitude occurs as the needle 828 passes the inductor of that circuit. The output voltage of each RLC circuit can be sensed as the needle 828 approaches and is advanced through the lumen 818. As the needle 828 enters and is advanced into the lumen 818, the inductance of each RLC circuit will change due to the presence of the needle 828. As shown in FIG. 9, the change in inductance will cause the output voltage of each RLC circuit to change—i.e. it will become non-resonant as the needle 828 passes by that inductor. The output voltage of each RLC circuit can thus be used as an indicator of the longitudinal position of the needle 828 with respect to the elongate member 808.

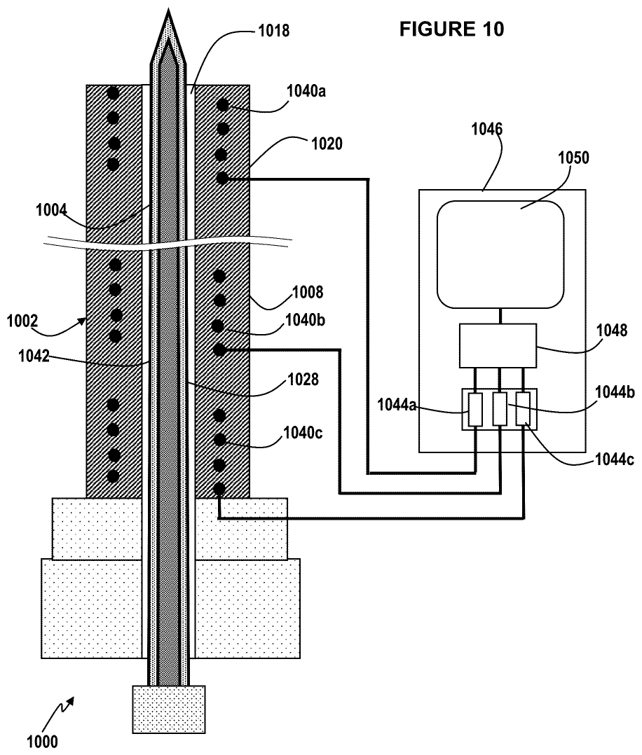

Referring now to FIG. 10, another alternative example system is shown. In the example of FIG. 10, features that are like those of FIGS. 1 to 3 will be referred to with like reference numerals, incremented by 900.

The system 1000 of FIG. 10 includes a first medical device 1002 in the form of a catheter having an elongate member 1008 with a lumen 1018, a second medical 1004 device including a needle 1028 that is advanceable through the lumen 1018, and a control unit 1046, which houses a processor 1048 and an output device 1050. The system 1000 of FIG. 10 is similar to the system 800 of FIG. 8, in that the system 1000 of FIG. 10 includes a plurality of RLC circuits (three of which are shown). Each RLC circuit includes an inductor that includes a coil that is embedded in the sidewall 1020 (i.e. the first RLC circuit includes a first coil 1040a; the second RLC circuit includes a second coil 1040b; and the third RLC circuit includes a third coil 1040c). The coils 1040a, 1040b, 1040c are longitudinally spaced apart so that the second coil 1040b is spaced proximally from the first coil 1040a, and the third coil 1040c is spaced proximally from the second coil 1040b.

The first RLC circuit further includes an excitation voltage source, a resistor, a capacitor, and an output voltage sensor, which are shown collectively at 1044a in FIG. 10; the second RLC circuit further includes an excitation voltage source, a resistor, a capacitor, and an output voltage sensor, which are shown collectively at 1044b in FIG. 10; and the third RLC circuit further includes an excitation voltage source, a resistor, a capacitor, and an output voltage sensor, which are shown collectively at 10044c in FIG. 8.

Similarly, to the system of FIG. 5, in the system of FIG. 10, the magnetic core 1042 of each inductor is provided by the needle 1028, which includes a coating of a magnetic material (e.g. a coating of epoxy containing suspended ferrites or magnetic tape).

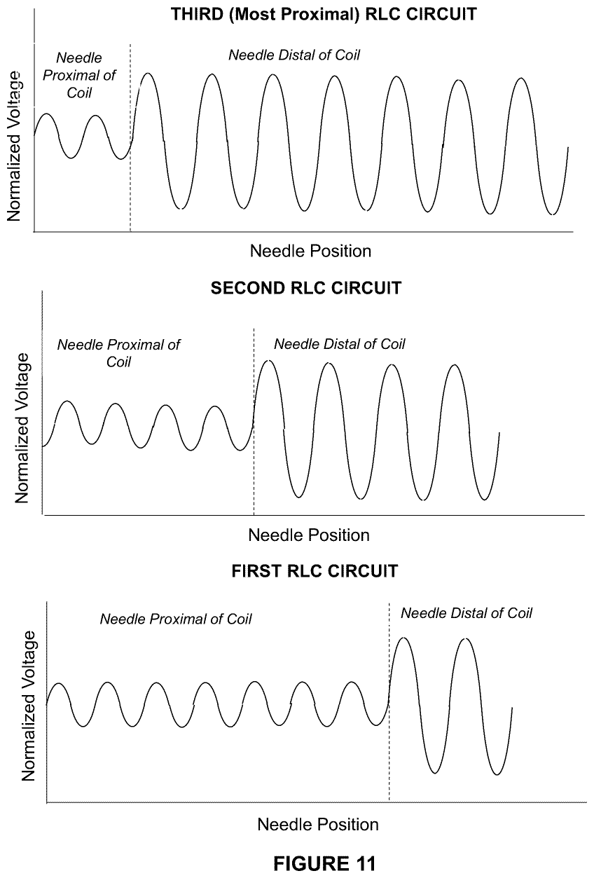

In use, an excitation voltage can be applied to each RLC circuit from the respective excitation voltage source as the needle 1028 approaches and is advanced through the lumen 1018. Each RLC circuit can be tuned so that the circuit becomes resonant as the needle 1028 passes the inductor of that circuit. The output voltage of each RLC circuit can be sensed as the needle 1028 approaches and is advanced through the lumen 1018. As the needle 1028 enters and is advanced into the lumen 1018, the inductance of each RLC circuit will change due to the presence of the needle 1028. As shown in FIG. 11, the change in inductance will cause the output voltage of each RLC circuit to change—i.e. it will become resonant as the needle 1028 passes by that inductor. The output voltage of each RLC circuit can thus be used as an indicator of the longitudinal position of the needle 1028 with respect to the elongate member 1008.

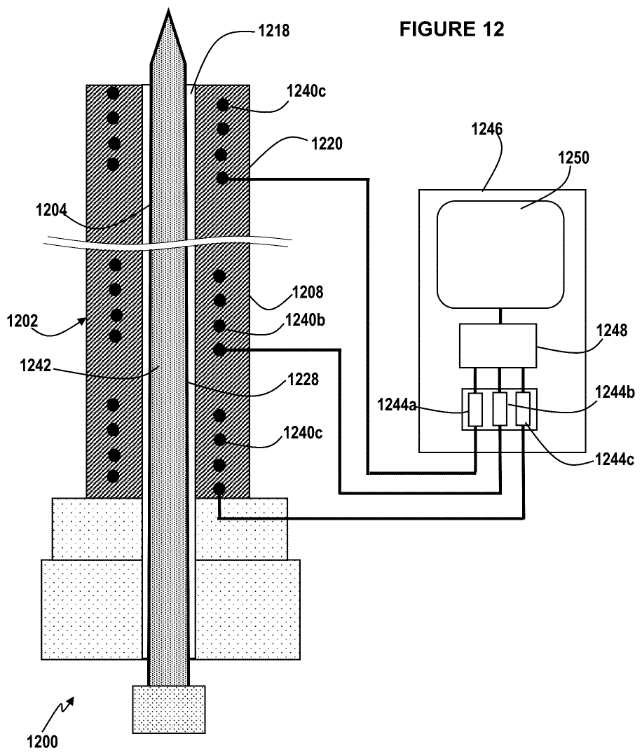

Referring now to FIG. 12, another alternative example system is shown. In the example of FIG. 12, features that are like those of FIGS. 1 to 3 will be referred to with like reference numerals, incremented by 1100.

The system 1200 of FIG. 12 is similar to the system 1000 of FIG. 10, and includes a first medical 1202 device in the form of a catheter having an elongate member 1208 with a lumen 1218, a second medical device 1204 including a needle 1228 that is advanceable through the lumen 1218, and a control unit 1246, which houses a processor 1248 and an output device 1250.

Similarly to the system 1000 of FIG. 10, the system 1200 includes a plurality of RLC circuits (three of which are shown). Each RLC circuit includes an inductor that includes a coil that is embedded in the sidewall 1220 (i.e. the first RLC circuit includes a first coil 1240a; the second RLC circuit includes a second coil 1240b; and the third RLC circuit includes a third coil 1240c). The coils 1240a, 1240b, 1240c are longitudinally spaced apart so that the second coil 1240b is spaced proximally from the first coil 1240a, and the third coil 1240c is spaced proximally from the second coil 1240b.

Similarly to the system 1000 of FIG. 10, each inductor includes a magnetic core 1242 that is provided by the needle 1228; however, in the system 1200 of FIG. 12, the needle is fabricated from a magnetic material, such as silicon steel, manganese-zinc ferrite, iron, etc, so that the needle 1228 as a whole is magnetic.

The system 1200 of FIG. 12 can be operated similarly to the system 1000 of FIG. 10—i.e. the excitation voltage can be tuned so that, when the needle 1228 is fully inserted into the elongate member 1208, the circuit is resonant, and the output voltage can thus be used as an indicator of the longitudinal position of the needle 1228 with respect to the elongate member 1208.

The devices and systems described above can be used in various medical procedures, but may be particularly useful in transseptal perforation procedures, in which a dilator (i.e. a first medical device) is advanced via the femoral vein towards the heart and positioned adjacent the fossa ovalis of the atrial septum, and then a transseptal perforation device (i.e. a second medical device) is advanced into and through the lumen of the dilator, from the proximal end of the dilator towards the distal end of the dilator. In such procedures, as the transseptal perforation device is advanced through the lumen of the dilator, an excitation voltage can be applied to the RLC circuit(s), and the output voltage of the RLC circuit(s) can be sensed and monitored, to provide an indicator of the longitudinal position of the transseptal perforation device with respect to the dilator.

As described above, an output can be generated based on the output voltage, to provide an indication of the longitudinal position of the transseptal perforation device with respect to the dilator. The output can be, for example, in the form of an image, or a light. This can help an operator to ensure that the perforating tip of the perforation device is shrouded within the dilator until it is ready for use by the operator.

Optionally, based on the output, the position of the transseptal perforation device can be adjusted with respect to the dilator. For example, if a red light illuminates before the user is ready to perforate the fossa ovalis, the user can withdraw the transseptal perforation device proximally, until a green light illuminates.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

What is claimed is:

1. A method of performing a transseptal perforation procedure, the method comprising:
providing a first medical device including an elongate member, the elongate member having a proximal portion defining a proximal end, a distal portion defining a distal end, and a lumen extending longitudinally therethrough from the proximal end to the distal end;
providing a second medical device including a radiofrequency perforation device, the radiofrequency perforation device having a proximal portion defining a proximal end and a distal portion defining a distal end;
advancing the distal end of the radiofrequency perforation device along the lumen toward the distal end of the elongate member causing a change in an electrical characteristic of a circuit associated with the first and second medical devices;
sensing, via a sensor in communication with the circuit, the change in an electrical characteristic of the circuit and generating a sensor signal based on the change in the electrical characteristic;
receiving, via a processor in communication with the sensor, the sensor signal; and
processing the sensor signal to generate a processor signal indicative of the longitudinal position of the distal end of the radiofrequency perforation device with respect to the distal end of the elongate member.

2. The method of claim 1, wherein the processor signal indicates that the distal end of the radiofrequency perforation device is adjacent a fossa ovalis of an atrial septum of a patient's heart.

3. The method of claim 1, wherein the processor signal indicates that the distal end of the radiofrequency perforation device has perforated the atrial septum.

4. The method of claim 1, wherein the electrical characteristic is inductance.

5. The method of claim 4, wherein the circuit is an RLC circuit comprising an inductor, a resistor, a capacitor, and an excitation voltage source.

6. The method of claim 5, wherein the inductor comprises a coil coupled with the elongate member.

7. The method of claim 5, wherein the inductor comprises at least a portion of the radiofrequency perforation device.

8. The method of claim 1, further comprising receiving the processor signal at an output device and generating an output based on the processor signal.

9. The method of claim 8, wherein the processor signal is configured to cause the output device to generate an image showing the longitudinal position of the radiofrequency perforation device within the elongate member.

10. The method of claim 8, wherein the processor signal is configured to cause illumination of a light of the output device.

11. A method of performing a transseptal perforation procedure, the method comprising:
advancing a first medical device via the femoral vein toward a heart of a patient, the first medical device including an elongate member, the elongate member having a proximal portion defining a proximal end, a distal portion defining a distal end, and a lumen extending longitudinally therethrough from the proximal end to the distal end;
advancing a second medical device along the lumen of the elongate member, the second medical device including a radiofrequency perforation device, the radiofrequency perforation device having a proximal portion defining a proximal end and a distal portion defining a distal end;
providing a circuit associated with the first and second medical devices, the circuit including a sensor configured to monitor the circuit for changes in electrical values and a processor in communication with the sensor;
positioning the distal end of the elongate member proximate a fossa ovalis of an atrial septum of the patient's heart and extending the distal end of radiofrequency perforation device beyond the distal end of the elongate member to perforate the atrial septum;
generating, via the sensor, a sensor signal indicative of a change in an electrical value and communicating the sensor signal to the processor; and
processing, via the processor, the sensor signal to generate a processor signal indicative of the longitudinal position of the distal end of the radiofrequency perforation device with respect to the distal end of the elongate member.

12. The method of claim 11, wherein the circuit comprises an RLC circuit and wherein the operation of advancing the second medical device comprises passing the second medical device through a coil of an inductor of the RLC circuit.

13. The method of claim 12, wherein the inductor comprises at least one coil disposed at the distal end of the elongate member.

14. The method of claim 11, further comprising transmitting the processor signal to an output device to generate an image or to illuminate a light.

15. The method of claim 14, further comprising adjusting a position of the radiofrequency perforation device with respect to the elongate member based on the generated image or illuminated light.

16. A system for performing a transseptal perforation procedure, the system comprising:
elongate member, the elongate member having a proximal portion defining a proximal end, a distal portion defining a distal end, and a lumen extending longitudinally therethrough from the proximal end to the distal end;
a radiofrequency perforation device, the radiofrequency perforation device having a proximal portion defining a proximal end and a distal portion defining a distal end, wherein the radiofrequency perforation device is configured to be advanced along the lumen of the elongate member toward the distal end of the elongate member; and
a circuit associated with the first and second medical devices, the circuit comprising a sensor and a processor in communication with the sensor;
wherein the sensor is configured to sense a change in an electrical characteristic of the circuit occurring when the distal end of the radiofrequency perforation device has passed beyond the distal end of the elongate member and punctured an atrial septum of a patient's heart and generate a sensor signal based on the change;
wherein the processor is configured to process the sensor signal to generate a processor signal indicative of the longitudinal position of the distal end of the radiofrequency perforation device with respect to the distal end of the elongate member.

17. The system of claim 16, wherein the circuit is an RLC circuit comprising an inductor, a resistor, a capacitor, and an excitation voltage source.

18. The system of claim 17, wherein the inductor comprises a coil that is supported by the elongate member and wound around the lumen.

19. The system of claim 16, further comprising an output device in communication with the processor and configured to receive the processor signal and generate an output based on the processor signal.

20. The system of claim 19, wherein the output device comprises at least one of:
a light, wherein the output comprises illumination of the light when the radiofrequency perforation device is at a predetermined longitudinal position with respect to the elongate member; and
a screen, wherein the output comprises a GUI showing an image of the longitudinal position of the radiofrequency perforation device with respect to the elongate member.
